(19) United States
(12) Patent Application Publication
(10) Pub. No.: US 2012/0128795 A1
(43) Pub. Date: May 24, 2012

(54) COLORLESS, NON-FUMING, HAZARDLESS AND STABLE AQUA-REGIA, ITS PREPARATION METHOD AND USE IN TREATING MEDICAL DISORDERS

(76) Inventor: John VINOTH, Madurai (IN)

(21) Appl. No.: 13/286,721

(22) Filed: Nov. 1, 2011

(30) Foreign Application Priority Data
Nov. 24, 2010 (IN) IN 3543/CHE/2010

(51) Int. Cl. A61K 33/00; A61P 1/12; B01J 19/00

(52) U.S. Cl. 424/666; 422/129

(57) ABSTRACT

A method for production of colorless, non-fuming, hazardless and stable aqua-regia solution is provided. The method includes heating a dry mixture of potassium nitrate, potassium chloride and potassium aluminum sulphate to obtain vapors of aqua-regia and condensing the vapors obtained to form a colorless, non-fuming, hazardless and stable aqua-regia solution. The method produces aqua-regia directly from dry salts without adding any single drop of solvent. Aqua-regia obtained by this method is highly stable and possesses dual property of being an anti-diarrheal as well as a rehydrating compound.

### BACKGROUND

1. Technical Field

The invention generally relates to a method for production of acid solutions, and more particularly to the production of colorless, non-fuming, hazardless and stable aqua-regia solution. The aqua-regia solution prepared using this process can be used for treatment of diarrhea.

2. Description of the Related Art

Nitric Acid (HN03) is a strong and corrosive acid. Generally in laboratories, the nitric acid is synthesized from copper (II) nitrate or by reacting equal masses of a nitrate salt with 96% sulphuric acid (H2S04), and distilling this mixture at boiling point of nitric acid i.e. 83° C. until only a white crystalline mass (a metal sulphate) remains in the reaction vessel. The red fuming nitric acid obtained may be converted to the white nitric acid. Hydrochloric acid is prepared by dissolving hydrogen chloride into water by an inverted funnel arrangement.

These conventional processes for producing nitric acid and hydrochloric acid are dangerous, hazardous and are not safe. These processes require solvents that are highly corrosive, highly reactive, strong oxidizers and poisonous. Further acid production by these conventional processes produce vapors that can cause breathing difficulties and may lead to pneumonia and pulmonary oedema, which may be fatal. Also swallowing of nitric acid or hydrochloric acid can cause immediate pain and burns of the mouth, throat, oesophagus and gastrointestinal tract. When brought in contact with skin, it can cause redness, pain, and severe skin burns. Concentrated nitric acid solution causes deep ulcers and stains on skin of a yellow or yellow-brown color. Its vapors are irritating and may permanently damage eyes. Long-term exposure to concentrated vapors of nitric acid may cause erosion of teeth and lung damage. People with pre-existing skin disorders, eye disease, or cardiopulmonary diseases may be more susceptible to the effects of this substance.

A mixture of Nitric acid and Hydrochloric acid in an approximate molar ratio of 1:3 is called as aqua-regia. Aqua-regia is ordinarily capable of dissolving gold, platinum, silver etc. Aqua-regia is also used in refining highest quality of gold and is also used in laboratories to clean glassware and other laboratory equipments. Conventionally, aqua-regia is prepared by mixing freshly prepared hydrochloric acid with nitric acid in molar ratio of about 1:3, however, preparation by this conventional method is very dangerous as it prone to bursting. Further it has to be prepared inside a fumehood. Also, aqua-regia prepared by conventional methods is highly unstable and due to a reaction between its components, it quickly loses its effect, further, aqua-regia is a highly corrosive chemical and causes severe burns to one handling it. Due to its high instability and corrosive properties, the application of aqua-regia in treating medical disorders has conventionally not been explored. Therefore there remains a need to develop a process for synthesis of hazardless and stable aqua-regia.

### SUMMARY

In view of the foregoing, an embodiment herein provides a process for production of a colorless, non-fuming, stable and hazardless aqua-regia solution. The process includes heating a dry mixture of a nitrate source, a chloride source and a sulphate source to obtain vapors of aqua-regia and condensing the vapors to form a colorless, non-fuming, stable and hazardless aqua-regia solution.

In another embodiment, a colorless, non-fuming, stable and hazardless aqua-regia is provided.

In yet another embodiment, a system for preparing aqua-regia is provided. The system comprises a reaction chamber, wherein a dry mixture of potassium nitrate, potassium chloride and potassium aluminum sulphate is added to said reaction chamber; a heating means for heating said dry mixture in said reaction chamber to produce vapors of aqua-regia; a condensing means for condensing said vapors to form aqua-regia solution; and a collecting means for collecting said aqua regia solution.

In yet another embodiment, a method for treatment of diarrhea is provided. The method includes administering a diarrhea patient a therapeutically effective amount of aqua-regia or a pharmaceutically acceptable salt comprising aqua-regia or solvate or derivative thereof.

In yet another embodiment, a new, hazardless and safe process for production of colorless, and non-fuming nitric acid solution is provided. The process includes heating a dry mixture of potassium nitrate and potassium aluminum sulphate to obtain vapors of nitric acid and condensing the vapors to form the colorless and non-fuming liquid nitric acid solution.

In yet another embodiment, a colorless and non-fuming nitric acid solution is provided.

In yet another embodiment, a new, hazardless and safe process for production of colorless and non-fuming hydrochloric acid solution is provided. The process includes heating a dry mixture of potassium chloride and potassium aluminum sulphate to obtain vapors of nitric acid and condensing said vapors to form the colorless and non-fuming liquid hydrochloric acid solution.

In yet another embodiment, a colorless and non-fuming hydrochloric acid solution is provided.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made embodiments herein include all such modifications.

### BRIEF DESCRIPTION OF THE DRAWINGS

The embodiments herein will be better understood from the following detailed description with reference to the drawings, in which:

FIG. 1 illustrates an apparatus for producing hazardless acid solutions and aqua-regia in accordance with an exemplary embodiment;

FIG. 2 illustrates a table illustrating the contents of hazardless and stable aqua-regia solution and their concentration according to an exemplary embodiment herein;

FIG. 3 illustrates a system for producing aqua-regia solution in accordance with an exemplary embodiment;

FIG. 4A-4B is a graphical representation illustrating a comparison of nitric acid solution produced by the hazardless process described in the invention with a conventional nitric acid solution according to an embodiment herein;

FIG. 5A-5B illustrates weight of the faeces recorded for three groups of animals to illustrate use of the aqua-regia in treatment of diarrhea, according to an exemplary embodiment herein;

FIG. 6A-6C illustrates results of gastrointestinal motility test conducted on three groups of animals to illustrate use of aqua-regia in rehydrating body cells, according to an exemplary embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

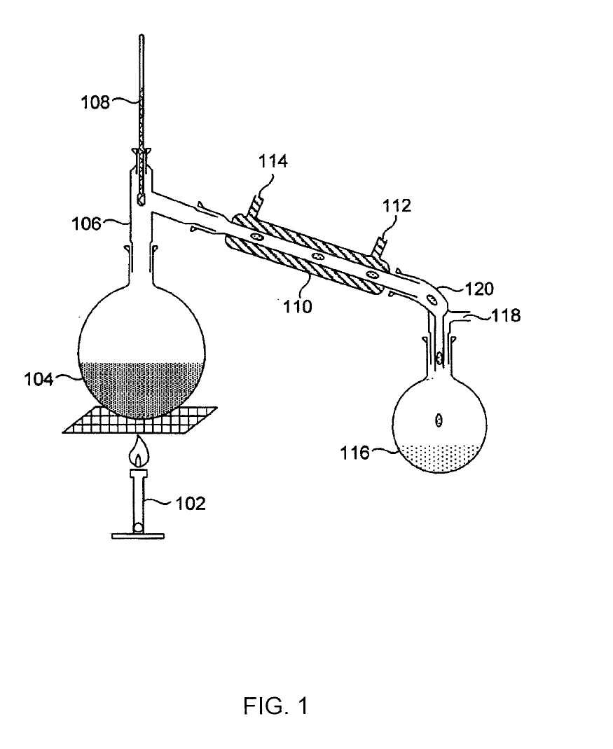

The present invention provides a process for synthesis of a colorless, non-fuming, stable and hazardless aqua-regia solution which can be used for treating medical disorders. The invention also provides a hazardless and safe process for synthesis of acid solutions such as, but not limited to, nitric acid and hydrochloric acid. Referring now to the drawing and more particularly to FIG. 1 where similar reference characters denote corresponding features consistently throughout the figures, there are shown preferred embodiments.

FIG. 1 illustrates an apparatus for producing a colorless, non-fuming, stable and hazardless aqua-regia solution according to an exemplary embodiment herein. The apparatus comprises a Bunsen burner 102, a round bottomed flask 104, a distillation adapter 106, a thermometer 108, a condenser 110, a water inlet 112, a water outlet 114, a connector 120, a vacuum adapter 118 and a collecting flask 116. A dry mixture of a chloride source such as, but not limited to, Potassium Chloride (KCl), a nitrate source such as, but not limited to, Potassium Nitrate (KN03) and a sulphate source such as, but not limited to, Potassium Aluminum Sulphate (KAl(S04)2) is placed in the round bottomed flask 104 without adding any solvent. The amount of Chloride source in the dry mixture can be in the range of 200-225 grams, the amount of Nitrate source can be in the range of 100-125 grams and the amount of Sulphate source can be in the range of 175-200 grams.

In an exemplary embodiment, the ratio of Chloride source, Nitrate source, and Sulphate source in the mixture is 200:125:175 respectively.

The Bunsen burner 102 heats the round bottomed flask 104 at 100° C. On heating, the dry mixture of chloride source (eg. Potassium Chloride (KCl)), nitrate source (eg. Potassium Nitrate (KN03)) and sulphate source (eg. Potassium Aluminum Sulphate (KAL(S04)2)) will react to produce vapors of aqua-regia which will be condensed by the condenser 110. The condenser 110 is connected to a water tap (not shown in figure) through the water inlet 112 to allow water to come in to the condenser and cool the aqua-regia vapors after which the warm water exits condenser through the water outlet 114. In the collecting flask 122 the condensed liquid aqua-regia solution is collected.

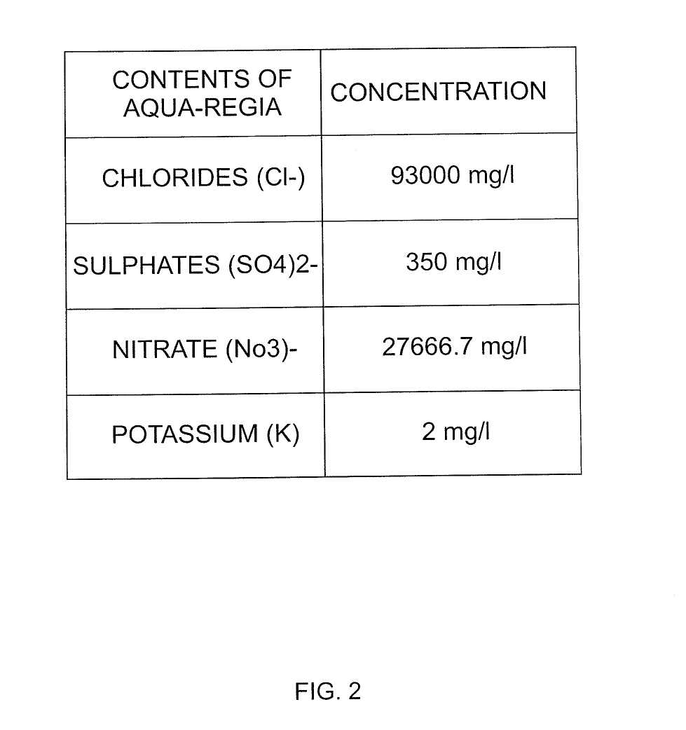

A series of confirmatory tests may be performed to confirm the presence of aqua-regia. In an exemplary embodiment, gold dissolves in the solution obtained by above method confirming the solution to be aqua-regia. In another exemplary embodiment, the contents and their concentration of the hazardless and stable aqua-reqia solution obtained by above method is determined by spectroscopic analysis (using spectroquant 118) and is illustrated in a table as shown in FIG. 2. In the solution, the Chlorides is present as Cl⁻ ions. The concentration of cl⁻ ions in the solution is 93000 mg/litre. Sulphate is present as [S0₄]²⁻ ions in the solution with a concentration of 350 mg/litre. Nitrate is present as [NO₃]⁻ ions in the solution with a concentration of 27666.7 mg/litre. Potassium is present in its elemental form as K in the solution with a concentration of 2 mg/litre.

The aqua-regia thus obtained is hazardless, non corrosive and stable. The aqua-regia obtained by above method may be preserved for several years without losing its effectiveness and any of its properties. The method described above produces aqua-regia directly from dry salts without adding any single drop of solvent and is thus safe, hazardless, require less man power and cost effective.

The aqua-regia produced by this method can be used as a therapeutic agent for medical treatments such as, but not limited to, treatment of diarrhea. A single dose of aqua-regia produced by above described method shows a remarkable anti-diarrheal activity evidenced by a reduction in the rate of defecation and a profound decrease in intestinal transit. Aqua-regia obtained by above method possesses dual property of being an anti-diarrheal as well as a rehydrating compound and when used as a drug, it has activity for at least 2 years.

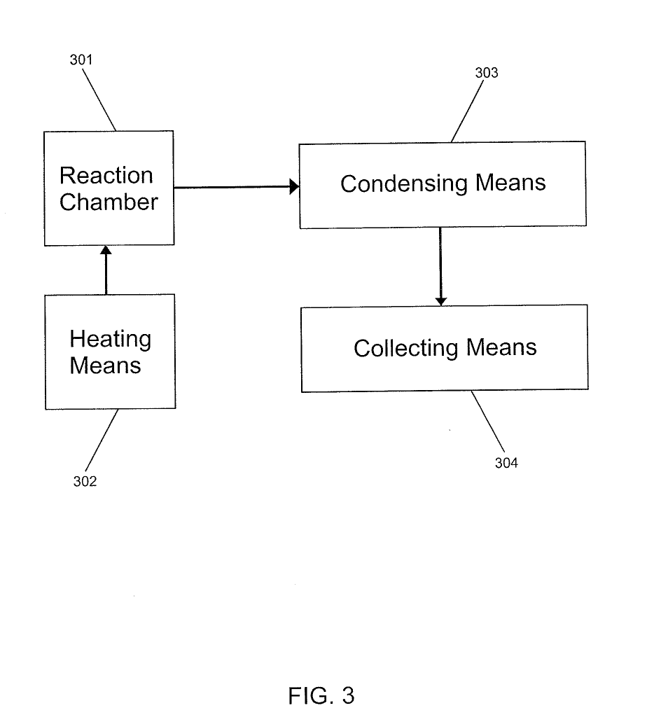

In yet another exemplary embodiment, a system for preparing aqua-regia is provided as shown in FIG. 3. The system comprises a reaction chamber 301, wherein a dry mixture of potassium chloride, potassium nitrate and potassium aluminum sulphate is added in a ratio of 8:5:7 respectively to said reaction chamber 301. The dry mixture is heated at 100° C. by a heating means 302 to produce vapors of aqua-regia. The aqua-regia vapors thus produced are condensed by a condensing means 303 to produce aqua-regia solution which is finally collected in a collecting means 304.

The heating means 302 can be any type of heating equipments that can heat the dry mixture at a temperature of 100° C. such as, but not limited to, burners, flames, candles, solar heaters, heating filaments or heating wires. The condensing means 303 can be any cooling means that can condense the vapors into solution such as, but not limited to, water jacket. The collection means 304 can be any vessel or container.

In yet another exemplary embodiment, a method of treatment of diarrhea is provided. The method includes administering to a patient a therapeutically effective amount of aqua-regia or a pharmaceutically acceptable salt comprising aqua-regia or a solvate or a derivative thereof wherein said aqua-regia is a colorless, non-fuming, hazardless and stable aqua-regia produced according to an exemplary embodiment of the invention described above.

In yet another exemplary embodiment, the apparatus shown in FIG. 1 is used for producing nitric acid solution. A dry mixture of equal amount of a nitrate source such as, but not limited to, Potassium Nitrate (KN03) and a sulphate source such as, but not limited to, Potassium Aluminum Sulphate (KAl(S04)2) is placed in the round bottomed flask 104 without adding any solvent. The Bunsen burner 102 heats the round bottomed flask 104 at 100° C. On heating, Potassium Nitrate (KN03) and Potassium Aluminum Sulphate (KAl(S04)2) will react to produce Nitric Acid (HN03) vapors which will be condensed by the condenser 110. The condenser 110 is connected to a water tap (not shown in figure) through the water inlet 112 to allow water to come in to the condenser and cool the nitric acid vapors after which the warm water exits condenser through the water outlet 114. In the collecting flask 122 the condensed liquid nitric acid solution is collected. The strength of nitric acid solution obtained by the process is 36.40% which can be further concentrated to obtain the strength of 100%.

A series of confirmatory test may be performed to determine the presence of nitrate in the nitric acid solution produced by the hazardless process described above. In an exemplary embodiment, the solution is reacted with ferrous sulphate (FeS04) in a test-tube, followed by slowly adding concentrated sulphuric acid into the reaction mixture which gives brown ring on the inside of test tube. The formation of the brown ring confirms the presence of nitrate ions in the nitric acid solution. The nitric acid solution obtained from above method is found to be non-corrosive and non-fuming and further since the process used for producing nitric acid involves dry salts without any solvent, the process is safe and hazardless.

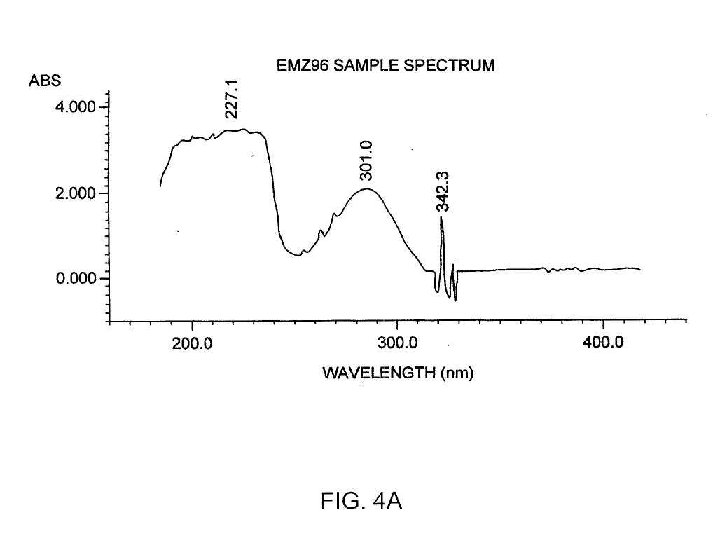

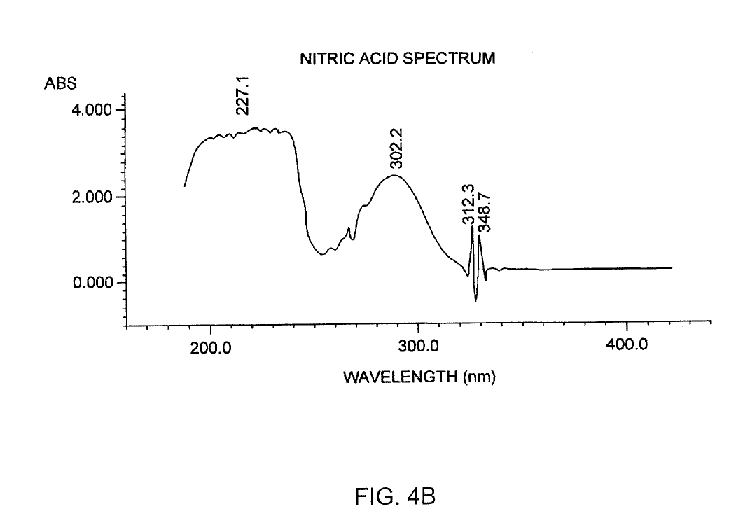

FIG. 4A-4B is a graphical representation illustrating a comparison of nitric acid solution obtained by the hazardless process described above with a conventional nitric acid solution according to an exemplary embodiment herein. The graph is obtained by measuring absorbance of UV-Visible light by both the solutions in a UV-Visible spectrophotometer at difference wavelengths of UV-Visible light. Absorbance of the UV-Visible light is plotted against the y-axis while the different wavelengths of the UV-Visible light are plotted against the x-axis. The sample of nitric acid solution obtained by the hazardless process is represented as EM296 and its absorbance at various wavelengths is shown in FIG. 4A. For the sample EM296,the highest absorbance of 3.2 is recorded at a wavelength of 227.1 nm which is equal to the absorbance obtained for the conventional nitric acid solution at same wavelength which is illustrated in FIG. 4B.

In yet another exemplary embodiment, the apparatus shown in FIG. 1 is used for producing hydrochloric acid solution, wherein a dry mixture of equal amount of a chloride source such as, but not limited to, Potassium Chloride (KCl) and a sulphate source such as, but not limited to, Potassium Aluminum Sulphate (KAl(S04)2) is placed in the round bottomed flask 104 without adding any solvent. On heating the round bottom flask 104 to 100° C. using Bunsen burner 102, Potassium Chloride (KCl) and Potassium Aluminum Sulphate (KAl(S04)2) react to produce Hydrochloric Acid (HCl) vapors which will be condensed by the condenser 110. The condensed hydrochloric acid vapors are collected in a collecting flask 122. The hydrochloric acid solution obtained is hazardless in nature. A series of confirmatory tests may be performed to determine presence of HCl in the hydrochloric acid solutions obtained by the hazrdless process described above. In an exemplary embodiment, the hydrochloric acid solution is reacted with sodium carbonate. Thereafter, a gas evolving from the reactant mixture is passed through lime water which turns the lime water cloudy confirming presence of hydrogen chloride (HCl) in the hydrochloric acid solution. The hydrochloric acid solution obtained from above method is found to be non-corrosive and non-fuming.

The process for producing aqua-regia and acids such as, but not limited to, nitric acid and hydrochloric acid, according to an exemplary embodiment described herein produces aqua-regia and acids directly from dry salts without adding any solvent. Therefore, the process is safe, hazardless, less challenging technically, more efficient and reduces overall cost as well.

The following examples are provided merely as illustrative of various aspects of the invention and shall not be construed to limit the invention in any way. In the following examples, it is to be understood that while efforts have been made to ensure the accuracy of the experimental parameters (e.g., amounts, temperature, etc.), some experimental error and deviation should be taken into account when reproducing the experiments set forth below.

### EXAMPLES

### Example 1

### Anti-Diarrheal Activity of Aqua-Regia

### A. Materials and Methods

### Experimental Animals

The anti-diarrheal activity of the hazardless and stable aqua-regia in terms of reduction of rate of defecation and consistency of faeces was tested on albino rats. In an embodiment, Swiss albino rats weighing 150-180 g of either sex were selected for the experiment. Animals were allowed to acclimatize for a period of 2 weeks in laboratory environment by housing them in a plurality of polypropylene cages. The polypropylene cages were maintained under standard laboratory conditions at an ambient temperature of 25° C. with 35%-60% humidity. Animals were fed with standard rat pellet diet.

### Chemicals and Reagents

Loperamide (standard reference anti-diarrheal drug), castor oil (laxative agent), and normal saline solution (0.9% NaCl) were used.

### Preparation of Aqua-Regia Drug

The aqua-regia was prepared by heating the dry mixture of three salts i.e. potassium nitrate (125 grams), potassium chloride (200 grams) and potassium aluminum sulphate (175 grams) in round bottom flask 104 without adding any solvent at a temperature of 100° C. for two hours. The salts will combine together and form vapors. That vapors will get condensed due to the water in the condenser 110. Then aqua regia will start to collect in the collecting flask 116 within 15 min after the process had started and it last for two hours to complete the process. The aqua-rega extracted was collected and stored. 0.5 ml of extracted aqua-regia was dissolved in 100 ml of distilled water to be used as dosage for treating diarrhea.

### Castor Oil-Induced Diarrhea

Experimental animals were kept on fasting for 18 hrs and divided into three groups of six animals per group. After fasting of 18 hrs, one of the groups labelled as control group receives normal saline (20 ml/kg i.p.), another group of the three receives the reference 10 mg/kg i.p. of the drug loperamide and is labelled as STD group. In the third group each animal receives a dose of 0.03 ml/rat p.o. of the aqua-regia as prepared by the method described above. The group is labelled as aqua-regia group. At one hour post treatment, 2 ml of castor oil was given orally to each animal and the rats were observed for defecation.

Animals of all the groups were placed separately in individual cages lined with filter paper. The filter paper was changed every 1 hour and the severity of diarrhea was assessed hourly for two hours. The total weight of faeces excreted were recorded within a period of two hours and compared with that of the control group. The total weight of diarrheal feces of the control group was considered 100%.

### Statistical Analysis

Data were analyzed by one-way ANOVA followed by Dunnett's t-test using computerized Graph Pad InStat version 3.05.

### B. Results

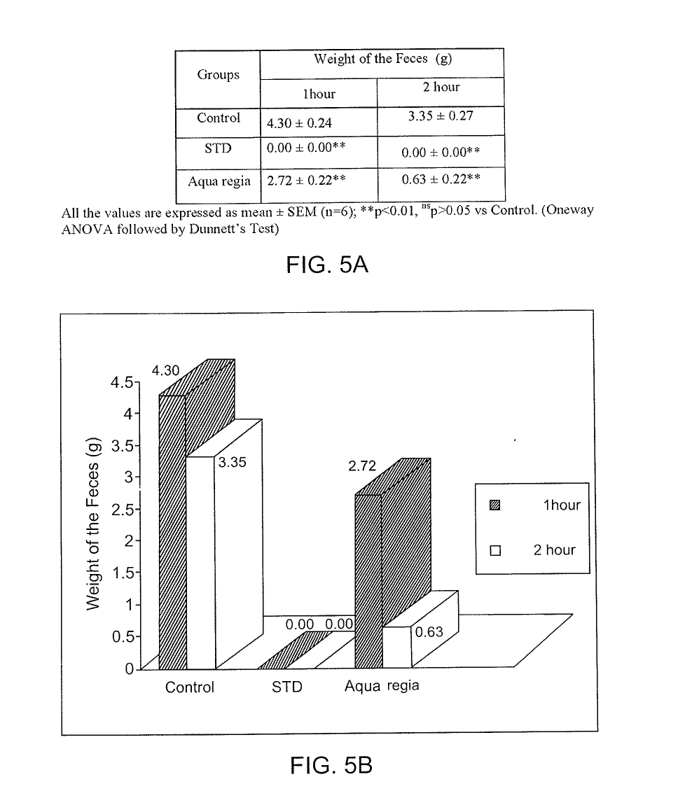

FIG. 5A-5B illustrates weight of the faeces recorded for three groups of animals to illustrate use of the aqua-regia in treatment of diarrhea, according to an exemplary embodiment herein. The weight of the faeces of the animals who received the dose of the aqua-regia considerably reduces from 2.72 g at the end of 1 hour to 0.63 g at the end of 2nd hour as compared to the control group illustrating its anti-diarrheal activity. As shown in FIG. 5A and 5B, it was observed that standard does not possess any diarrheal symptoms because usually loperamide causes constipation. Aqua-regia reduced the rate of defecation to almost half of that of the control in the first hour and in the second hour it almost became to 0 like standard group animals. This shows that it not only treats the symptoms of diarrhea but also the causes of diarrhea.

### Example 2

### Rehydrating Property of Aqua-Regia

### A. Materials and Methods

### Experimental Animals

The use of the hazardless and stable aqua-regia in rehydrating body cells was tested on albino rats. In an embodiment, Swiss albino rats weighing 150-180 g of either sex were selected for the experiment. Animals were allowed to acclimatize for a period of 2 weeks in laboratory environment by housing them in a plurality of polypropylene cages. The polypropylene cages were maintained under standard laboratory conditions at an ambient temperature of 25° C. with 35%-60% humidity. Animals were fed with standard rat pellet diet.

### Chemicals and Reagents

Atropine sulphate (standard reference anti-diarrheal drug), charcoal meal (10% activated charcoal in 5% gum acacia) and vehicle (2% v/v Tween 80 in distilled water) were used.

### Preparation of Aqua-Regia Drug

The aqua-regia was prepared by heating the dry mixture of three salts i.e. potassium nitrate (125 grams), potassium chloride (200 grams) and potassium aluminum sulphate (175 grams) in round bottom flask 104 without adding any solvent at a temperature of 100° C. for two hours. The salts will combine together and form vapors. That vapors will get condensed due to the water in the condenser 110. Then aqua-regia will start to collect in the collecting flask 116 within 15 min after the process had started and it last for two hours to complete the process. The aqua-regia extracted was collected and stored. 0.5 ml of extracted aqua-regia was dissolved in 100 ml of distilled water to be used as dosage for treating diarrhea.

### Gastrointestinal Motility Test

The gastrointestinal motility test was conducted to illustrate use of aqua-regia in rehydrating body cells, according to an embodiment herein. This test was conducted by using charcoal meal as a diet marker. The rats were divided into three equal groups of six animals each and kept on fasting for eighteen hours before the test. Each animal in the first group labelled as control group was orally administered a dose of 0.5% Tween 80 in distilled water. Each animal in second group received a dose of 0.1 mg/kg of bodyweight of the standard drug atropine sulphate. The group receiving standard drug was STD group. Each animal of the third group received a dose of 0.03 ml of aqua-regia. The group receiving aqua-regia was aqua-regia group. After one hour, each animal of all the groups was given 1 ml of charcoal meal (10% activated charcoal in 5% gum acacia) orally and were sacrificed thirty minutes after administration of the charcoal meal. The distance travelled by the charcoal meal was calculated. The distance covered by the charcoal meal in the intestine was expressed as a percentage of the total distance travelled from the pylorus to the caecum of each animal.

### Statistical Analysis

Data were analyzed by one-way ANOVA followed by Dunnett's t-test using computerized Graph Pad InStat version 3.05.

### B. Results

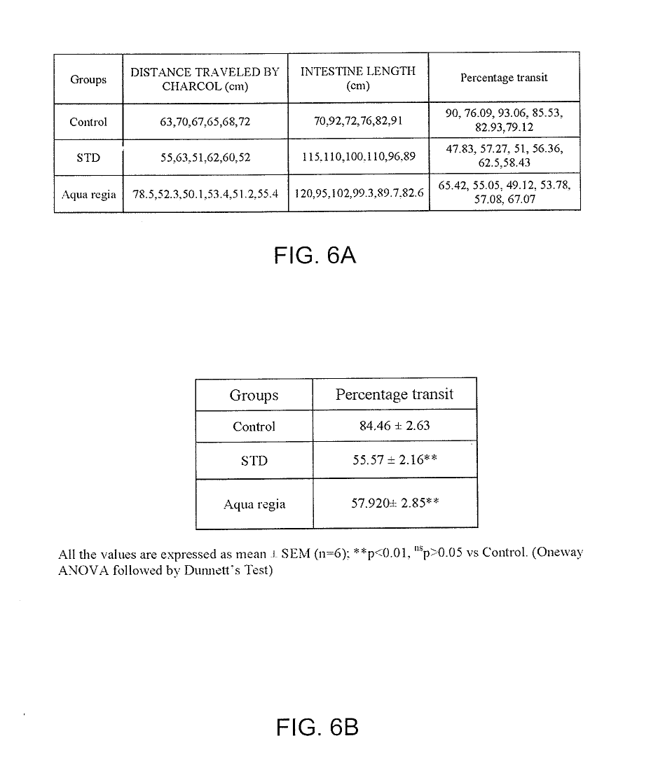

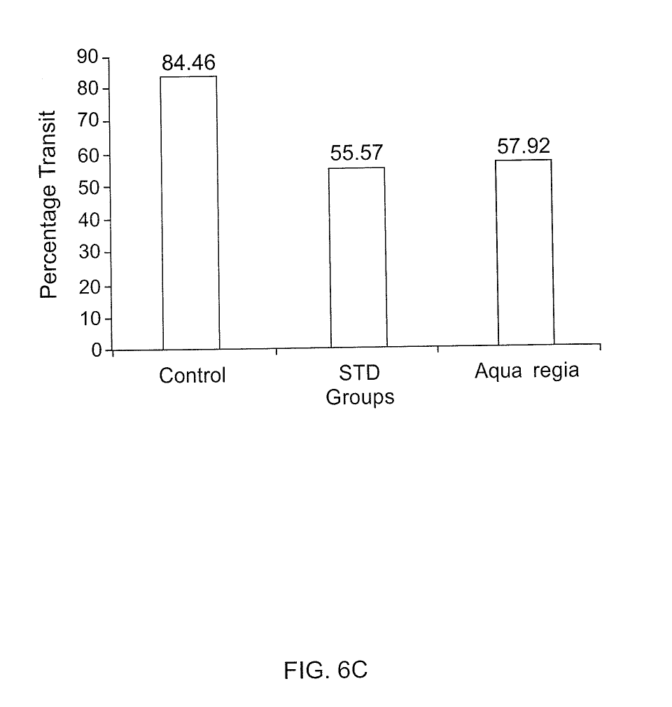

Results of the gastrointestinal motility test are shown in FIG. 6A, 6B and 6C. Aqua-regia significantly reduces intestinal transit as observed by the decrease in transit motility of charcoal meal. This may be due to the fact that the aqua-regia increases the re-absorption of water by decreasing intestinal motility. The third group which received the dose of the aqua-regia has percentage transit reduced to 57.9% as compared to 55.57% of the group which received standard drug and 84.46% of the control group.

Previous examples are provided to illustrate but not to limit the scope of the claimed inventions. Other variants of the inventions will be readily apparent to those of ordinary skill in the art and encompassed by the appended claims. All publications, patents, patent applications and other references cited herein are hereby incorporated by reference.

I claim:

1. A process for production of aqua-regia, said process comprising:
heating a mixture of a nitrate source, a chloride source and a sulphate source to produce vapors of aqua-regia; and
condensing said vapors to form said aqua regia.

2. The process of claim 1, wherein said mixture is heated at 100° C. to produce said vapors.

3. The process of claim 1, wherein at least one of said nitrate source, said chloride source and said sulphate source are dry without any solvent.

4. The process of claim 1, wherein said nitrate source is potassium nitrate, said chloride source is potassium chloride and said sulphate source is potassium aluminum sulphate.

5. The process of claim 1, wherein ratio of said nitrate source, said chloride source and said sulphate source is 5:8:7 respectively.

6. The process of claim 1, wherein said aqua-regia is used for treating medical disorders.

7. The process of claim 1, wherein said aqua-regia is stable for at least 2 years to be used as a therapeutic agent.

8. The process of claim 1, wherein said aqua-regia is non-fuming and hazardless.

9. A process for production of aqua-regia, said process comprising:
heating a dry mixture of potassium nitrate, potassium chloride and potassium aluminum sulphate to obtain vapors of aqua-regia; and
condensing said vapors to form said aqua-regia.

10. The process of claim 9, wherein said mixture is heated at 100° C. to produce said vapors.

11. The process of claim 9, wherein ratio of said potassium nitrate, said potassium chloride and said potassium aluminum sulphate in said mixture is 5:8:7 respectively.

12. The process of claim 9, wherein said aqua-regia is used for treating medical disorders.

13. The process of claim 9, wherein said aqua-regia is stable for at least 2 years to be used as a therapeutic agent.

14. The process of claim 9, wherein said aqua-regia is non-fuming and hazardless.

15. An aqua-regia prepared by method comprising:
heating a dry mixture of potassium nitrate, potassium chloride and potassium aluminum sulphate to obtain vapors of aqua-regia; and
condensing said vapors to form said aqua-regia.

16. The aqua-regia of claim 15, wherein said mixture is heated at 100° C. to produce said vapors.

17. The aqua-regia of claim 15, wherein ratio of said potassium nitrate, said potassium chloride and said potassium aluminum sulphate in said dry mixture is 5:8:7 respectively.

18. The aqua-regia of claim 15, wherein said aqua-regia is used for treating medical disorders.

19. The aqua-regia of claim 15, wherein said aqua-regia is stable for at least 2 years to be used as a therapeutic agent.

20. The aqua-regia of claim 15, wherein said aqua-regia is non-fuming and hazardless.

21. A method for treatment of diarrhea comprising:
administering to a patient a therapeutically effective amount of aqua-regia or a pharmaceutically acceptable salt comprising aqua-regia or a solvate or a derivative thereof wherein said aqua-regia is prepared by method comprising:
heating a dry mixture of potassium nitrate, potassium chloride and potassium aluminum sulphate to obtain vapors of aqua-regia; and
condensing said vapors to form said aqua-regia.

22. The method of claim 21, wherein ratio of said potassium nitrate, said potassium chloride and said potassium aluminum sulphate in said dry mixture is 5:8:7 respectively.

23. The method of claim 21, wherein said aqua-regia is stable for at least 2 years to be used as a therapeutic agent.

24. The method of claim 21, wherein said aqua-regia is non-fuming and hazardless.

25. A system for preparing aqua-regia solution, said system comprising:
a reaction chamber, wherein a dry mixture of potassium nitrate, potassium chloride and potassium aluminum sulphate is added to said reaction chamber;
a heating means for heating said dry mixture in said reaction chamber to produce vapors of aqua-regia;
a condensing means for condensing said vapors to form said aqua-regia solution; and
a collecting means for collecting said aqua regia solution.

26. The system of claim 25, wherein said dry mixture is heated at 100° C. to produce said vapors.

27. The system of claim 25, wherein ratio of said potassium nitrate, said potassium chloride and said potassium aluminum sulphate in said dry mixture is 5:8:7 respectively.

28. The system of claim 25, wherein said aqua-regia is stable for at least 2 years to be used as a therapeutic agent.

29. The system of claim 25, wherein said aqua-regia is non-fuming and hazardless.
